(19) United States
(12) Patent Application Publication
(10) Pub. No.: US 2026/0295256 A1
(43) Pub. Date: Oct. 1, 2026

(54) VISUALIZATION SYSTEM FOR REAL-TIME MONITORING OF THE COCHLEA

(71) Applicant: Northwestern University, Evanston, IL (US)

(72) Inventors: Claus-Peter Richter, Skokie, IL (US); Joaquin Cury, Chicago, IL (US)

(21) Appl. No.: 19/479,075

(22) PCT Filed: Apr. 25, 2024
(86) PCT No.: PCT/US2024/026207

§ 371 Date: Oct. 27, 2025

Related U.S. Application Data
(60) Provisional application No. 63/462,090, filed on Apr. 26, 2023.

(51) Int. Cl. A61N 1/36; A61B 34/20; A61B 90/00; A61N 1/05

(52) U.S. Cl. CPC A61N 1/36039; A61B 34/20; A61N 1/0541; A61B 2034/2057; A61B 2034/2065; A61B 2090/373

(57) ABSTRACT

A cochlea implant monitoring system includes a microcamera mounted to an electrode array of a cochlea implant. The system also includes a processor operatively coupled to the microcamera and configured to control the microcamera to capture images during insertion of the electrode array. The system also includes a display operatively coupled to the processor and configured to display the captured images in real-time during insertion of the electrode array.

### CROSS-REFERENCE TO RELATED APPLICATION

The present application claims the priority benefit of U.S. Provisional Patent App. No. 63/462,090 filed on Apr. 26, 2023, the entire disclosure of which is incorporated herein by reference.

### REFERENCE TO GOVERNMENT RIGHTS

This invention was made with government support under grant numbers DC018666 and DC017492 awarded by the National Institutes of Health. The government has certain rights in the invention.

### BACKGROUND

A cochlear implant refers to a device that enables individuals with severe-to profound hearing loss to perceive sounds. The cochlear implant is a surgically implanted neuroprosthesis that is used to stimulate the auditory nerve of a patient. The stimulation is generally performed by way of electrodes that are placed in the cochlea of the inner ear.

Specifically, the electrodes are activated in response to audio signals that are received by a microphone attached to the outer ear of the patient. The received audio signals are provided to an implanted receiver, which in turn activates the electrodes to stimulate the auditory nerve. Stimulation of the auditory nerve directs signals to the brain, which the brain interprets as sound.

### SUMMARY

An illustrative cochlea implant monitoring system includes a microcamera mounted to an electrode array of a cochlea implant. The system also includes a processor operatively coupled to the microcamera and configured to control the microcamera to capture images during insertion of the electrode array. The system also includes a display operatively coupled to the processor and configured to display the captured images in real-time during insertion of the electrode array.

The system can also include a video bridge chip coupled to the microcamera, where the video bridge chip includes an integrated circuit that performs image processing.

The video bridge chip can also include an analog-to-digital converter (ADC) to convert captured analog data to digital data. In one embodiment, the processor comprises a microcontroller that includes a digital camera module interface (DCMI). In another embodiment, the processor comprises a microcontroller that includes one or more of a parallel interface and a serial peripheral interface. In an illustrative embodiment, the microcamera is mounted to a distal end of the electrode array.

In another embodiment, a sampling frequency of the display is greater than or equal to a frame capture rate of the microcamera. In one embodiment, the display comprises a liquid crystal display (LCD). The system can also include one or more microlenses mounted to the microcamera, where the one or more microlenses are produced by curing one or more droplets of a polymer with ultraviolet (UV) light. Alternatively, the one or more microlenses can be in the form of a printed transparent polymer. In another embodiment, a polarizer is positioned in front of a camera chip of the microcamera, and a light source that emits polarized light toward the camera chip such that birefringent activity can be monitored by the processor. In an alternative embodiment, the system includes a waveguide in the form of an optical fiber or a polymer, and the waveguide directs polarized light toward the camera chip such that birefringent activity can be monitored by the processor.

An illustrative method of monitoring a cochlear implant during insertion includes controlling, by a processor, a microcamera mounted to an electrode array of the cochlear implant. The method also includes capturing, by the microcamera, images during insertion of the electrode array into a cochlea. The method further includes displaying, by a display operatively coupled to the processor, the captured images in real-time during insertion of the electrode array.

The method can also include controlling a frame capture rate of the microcamera to be less than or equal to a sampling frequency of the display. The method can further include emitting polarized light toward the microcamera, and the processor monitors birefringent activity that results from the polarized light. The polarized light can be emitted from a light source proximate to the microcamera. Alternatively, the polarized light is emitted from a waveguide in the form of an optical fiber or a polymer. The method can also include mounting one or more microlenses to the microcamera, where the one or more microlenses are produced by curing one or more droplets of a polymer with ultraviolet (UV) light. In an illustrative embodiment, the microcamera is incorporated into an electrode of the electrode array.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts an electrode array positioned in the cochlea in accordance with an illustrative embodiment.

FIG. 2 is a block diagram that depicts components of the cochlear implant visualization system in accordance with an illustrative embodiment.

FIG. 3 depicts the microcamera of the proposed system relative to a finger for size comparison in accordance with an illustrative embodiment.

FIG. 4 depicts a computing system for monitoring a cochlea implant procedure in accordance with an illustrative embodiment.

FIG. 5 is a flow diagram depicting operations performed to make and use a cochlear implant monitoring system in accordance with an illustrative embodiment.

### DETAILED DESCRIPTION

A cochlear implant (CI) is a small, complex electronic device that is used to restore some hearing for severely-to-profoundly deaf individuals. Traditional cochlear implants include an external component including a microphone, a speech processor, and a transmission coil. The device also includes an internal component that includes an implanted receiver coil, a stimulation unit, and a cochlear electrode array. The electrode array is surgically inserted into the cochlea conduit to electrically stimulate the auditory nerve.

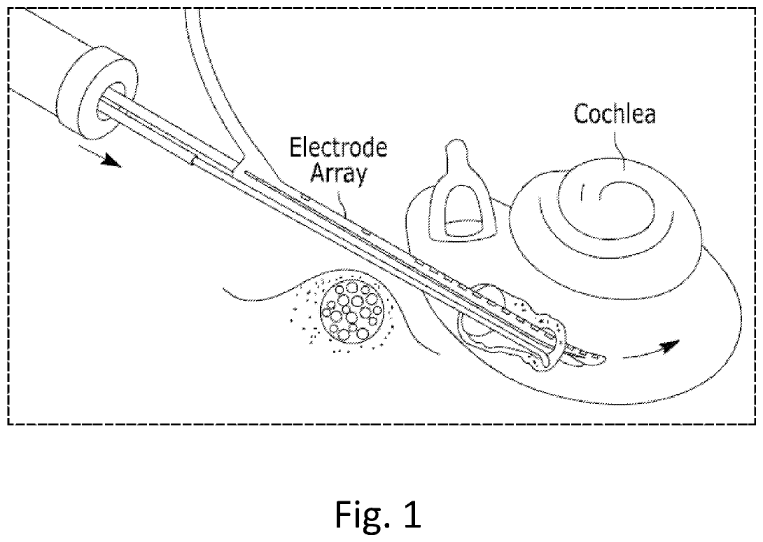

Specifically, in response to sounds captured by the microphone, the stimulation unit is controlled to activate electrodes in the electrode array to stimulate the auditory nerve, thereby mimicking the captured sound. FIG. 1 depicts an electrode array positioned in the cochlea in accordance with an illustrative embodiment.

Cochlear implants have been a significant breakthrough in the treatment of hearing loss and they have been successful in restoring speech understanding in patients with partial and complete deafness. One of the critical steps in the cochlear implantation process is the insertion of the electrode array into the cochlea because the insertion determines the quality and effectiveness of the electrical stimulation delivered to the auditory nerve.

Although CIs are one of the most successful neural prostheses, the surgery for inserting the electrode array poses a risk due to the complex anatomy of the inner ear, the delicate structures involved, and the variations in the anatomy between and within individuals.

The surgical procedure to implant an electrode array involves the insertion of the electrode array through a small incision in the cochlea, which requires precise manipulation of the array within a confined space. If the insertion trajectory of the electrode array does not perfectly match the shape of the scala tympani (ST), the electrode can traumatize the thin walls of the ST (the bony osseous spiral lamina (OSL) and the soft basilar membrane) or even translocate into one of the other two compartments (the scala vestibuli or the scala media) of the ear. As a result, improper insertion can cause several complications such as inflammation and the potential for loss of residual hearing. The latter complication has been determined to occur in up to 32% of implantations.

As a result of the high complication rate, atraumatic electrode insertion has received a surge of interest amongst CI surgeons and researchers in recent years in order to optimize hearing preservation. To reduce the risk of hearing loss in patients with residual hearing, several strategies have been developed, such as using smaller and more flexible electrodes, monitoring the insertion depth, and using image-guided surgery. Moreover, advances in surgical techniques and imaging technology have enabled surgeons to perform more precise and safer surgeries, reducing the risk of intraoperative trauma and hearing loss.

Close monitoring and follow-up care are also crucial in achieving a successful outcome for patients undergoing cochlear implantation. Monitoring techniques provide valuable information during the electrode insertion process and can help to ensure that the electrode is placed in the optimal position within the cochlea. The use of real-time monitoring techniques allows the surgical team to assess the insertion depth, angle, and force of the electrode as it is being inserted, which can improve the accuracy of placement and minimize the risk of damage to the delicate structures of the inner ear. By incorporating monitoring techniques into the surgical procedure, clinicians can help to ensure the best possible outcome for their patients.

Available monitoring techniques for electrode insertion into the cochlea include electrical impedance measurement, cone-beam computed tomography, fluoroscopy, measurement of insertion forces, cochlea response telemetry, and optical coherence tomography. Each of these monitoring techniques is briefly described below.

Electrical impedance measurement is a technique employed during cochlear implantation surgery to estimate the depth of insertion of the electrode array into the cochlea. This method involves measuring the impedance, or electrical resistance, between the electrode array and the cochlear tissues using a surgical instrument. The electrical impedance changes as the electrode array is inserted into the cochlea, allowing the surgeon to monitor the depth of insertion in real-time. The information provided by intraoperative electrical impedance measurement includes the estimation of the depth of insertion, which is essential for ensuring proper electrode positioning and optimal hearing outcomes for the patient. The technique is particularly useful for cases where there may be anatomical variations in the cochlea, making it difficult to accurately estimate the insertion depth based on anatomical landmarks alone. However, there are some limitations with the use of electrical impedance measurements. For example, this technique can be adversely affected by tissue edema, variations in tissue impedance, and/or electrode malfunction.

Cone-beam computed tomography (CBCT) is a technique that uses a cone-shaped X-ray beam to produce three-dimensional (3D) images of the cochlea and the electrode array. The CBCT technique provides information on the position and orientation of the electrode array relative to the cochlear anatomy as well as the depth of insertion. This technique generates high spatial resolution 3D images using a low radiation dose and short scan times. However, the resolution can decrease towards the edges of the images due to the nature of the cone-shaped beam used to generate the images.

Fluoroscopy is a monitoring technique in which a continuous x-ray beam is used to create real-time moving images of the inside of the body. Fluoroscopy is often used during medical procedures to guide the placement of instruments or to visualize the movement of contrast agents through organs or blood vessels. During cochlear implant surgery, fluoroscopy can be used to ensure that the electrode array is inserted into the correct position within the cochlea and to monitor the placement of the electrode during the procedure.

Insertion forces measurement refers to a procedure that involves measuring the insertion forces applied during cochlear implantation surgery. This is performed by attaching a strain gauge to the implant insertion tool, which measures the forces applied during insertion. The information provided by this technique is useful for ensuring that the electrode is being inserted with appropriate force and can help prevent trauma to the delicate structures of the inner ear. Additionally, this technique can provide information about any resistance encountered during insertion, which can indicate potential issues with the insertion path or anatomy.

Cochlea response telemetry is a technique that involves conducting electrocochleography (ECochG) directly from the electrode array during its insertion and relating temporal changes in the cochlear neural responses to surgical maneuvers. By monitoring the ECochG during the insertion process, surgeons can gain insight into the functional status of the cochlea and adjust their technique to optimize hearing outcomes for the patient. This technique has the potential to improve hearing outcomes and reduce the risk of intraoperative trauma to the cochlea. Studies using this technique indicate that the electrode array can be inserted to 20 millimeters (mm) without evidence of trauma. However, beyond this distance, the array should be advanced with meticulous care, very slowly, not against resistance, and in a manner that does not cause sudden movement of the array.

Optical coherence tomography (OCT) is a methodology that uses light to create high-resolution, cross-sectional images of biological tissues. OCT works by splitting a low-power, near-infrared light source into two beams: a sample beam and a reference beam. The sample beam is directed onto the tissue of interest (cochlea walls), while the reference beam is directed onto a mirror. The two beams then recombine, creating an interference pattern that can be measured and analyzed to generate an image of the cochlea. The method allows for monitoring in real-time of any potential trauma or damage to the delicate structures of the cochlea during the insertion process. Surgeons using OCT can adjust the orientation of the electrode array, so it does not come in contact with the cochlear walls, reducing risk of trauma. While OCT is promising, it has some inherent limitations. For instance, light wavelengths typically used in this technique have a limited penetration depth (e.g., 1-1.5 mm) due to the scattering and absorption of light in the tissues. Additionally, optical fibers used in this technique are not flexible enough to image the turn(s) of the cochlea structures.

In addition to the aforementioned monitoring methodologies, there is also growing interest in the use of robotics to assist with electrode insertion during cochlear implantation. While most electrode insertions are currently performed manually, robotic systems have the potential to provide greater accuracy and precision during the procedure. By using robotic assistance combined with monitoring techniques, surgeons may be able to achieve more consistent and optimal electrode placement, which could ultimately lead to improved hearing outcomes for patients. However, while robotic electrode insertion based on preoperative imaging may reduce the risk of electrode translocation, the value of the preoperative imaging is limited. In particular, the resolution of current preoperative imaging techniques, such as CT and fluoroscopy is too low to visualize the thin cochlear walls, which is necessary for atraumatic trajectory planning.

Additionally, although OCT shows promising results, it requires flexible waveguides to navigate the cochlear turns; otherwise, the technique is limited to a few millimeters at the beginning of the cochlea conduit. Intraoperative X-ray and transimpedance measurements can help detect electrode fold-over, but do not prevent it and provide no information on the actual position of the electrode inside the ST with respect to the cochlear walls at risk for trauma.

Thus, inserting an electrode into the cochlea is a delicate procedure, and it is important for the surgeon to ensure that the electrode is positioned precisely to stimulate the auditory nerve, while avoiding damage to the cochlear structures. As discussed above, although there are several monitoring techniques that can facilitate the insertion during this procedure, these techniques have limitations such that the surgeon often relies primarily on tactile feedback to determine the trajectory of insertion. Using such techniques, increased resistance can only be detected after the electrode has already touched the cochlear wall, potentially causing trauma. Furthermore, studies report that electrode insertion trauma is often not accompanied by an increased resistance or mechanical forces, making it currently an unreliable parameter for insertion monitoring.

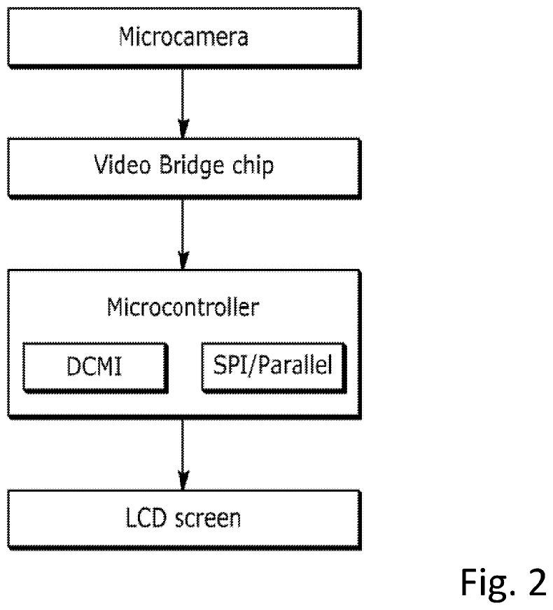

Described herein is a visualization system for real-time monitoring of electrode array insertion into the cochlea. In an illustrative embodiment, the proposed system can include a microcamera that operates in the visible range, a video bridge chip, a microcontroller including a digital camera module interface (DCMI) and serial peripheral interface (SPI)/parallel ports, and a peripheral device to visualize in real-time pictures and videos captured by the microcamera. FIG. 2 is a block diagram that depicts components of the cochlear implant visualization system in accordance with an illustrative embodiment. Components of the system are described in more detail below. In alternative embodiments, the cochlear implant visualization system can include fewer, additional, and/or different components.

Microtechnologies, also known as microelectromechanical systems (MEMS), refer to the technology of miniaturizing devices and systems to the micrometer scale. With advancements in microfabrication techniques, it has become possible to manufacture smaller sensors such as accelerometers, gyroscopes, pressure sensors, and temperature sensors, among others that are now widely used in various applications, ranging from smartphones to medical devices. By using MEMS techniques, it is possible to fabricate sensors with high precision and sensitivity, while keeping their size extremely small. For instance, microcameras with a size of only a few millimeters are now commercially available, making them suitable for several medical applications such as endoscopy. The small size of these image sensors also allows for their integration into other systems, making them more versatile and efficient.

In an illustrative embodiment, the microcamera used in in the proposed system is the OVM6948 (Omnivision, USA). It is one of the smallest cameras in the world, measuring just 0.575 mm×0.575 mm and providing a resolution of 200×200 pixels. In alternative embodiments, a different type of camera (e.g., the OCHT10 from Omnivision) may be used and/or the camera can have a different size or resolution. For example, in one embodiment, the camera used by the system can be ~50% or more smaller than the OVM6948 camera described above, and can have improved resolution as compared to the OVM6948.

Regardless of which type of camera is used, the micro-camera captures high-quality images. The camera chip used in the microcamera can also capture video at up to 30 frames per second (fps), providing smooth and seamless video playback. Alternatively, a different video capture rate may be used such 60 fps, 120 fps, etc. Despite its small size, the microcamera features advanced imaging technology, including microlenses and back-illumination, which helps to improve image quality and sensitivity in low-light conditions. The camera chip is also designed to be power-efficient, with a low power consumption of just 2 milliwatts, making it suitable for use in battery-powered devices. In alternative embodiments, the camera can have a different power consumption that is less than or greater than 2 milliwatts.

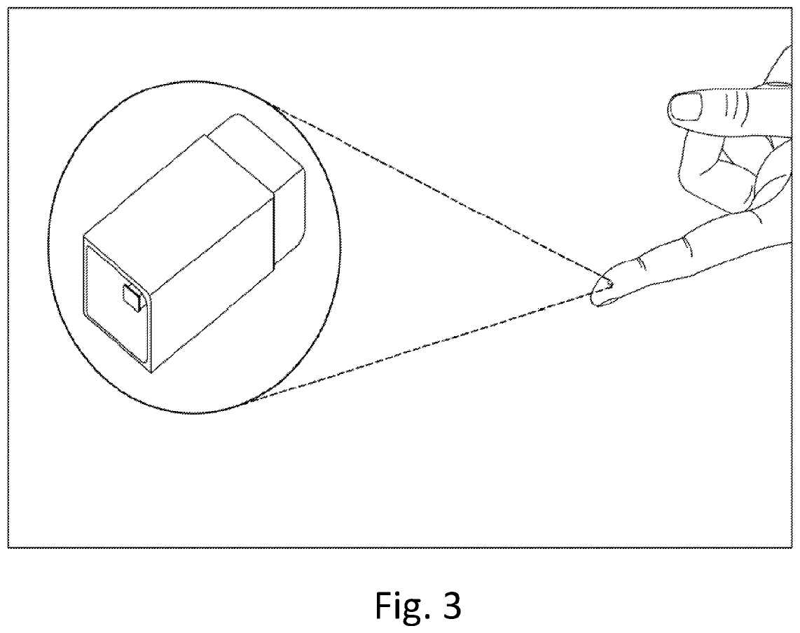

FIG. 3 depicts the microcamera of the proposed system relative to a finger for size comparison in accordance with an illustrative embodiment. FIG. 3 also includes a blown-up inset view of the microcamera. Due to its small size, the camera is a suitable option for integration with conventional cochlear electrode arrays. In one embodiment, the camera is positioned to form the tip (i.e., distal end) of the electrode array, allowing surgeons to visualize the cochlea walls during the insertion procedure. In another embodiment, the camera is incorporated into the body of the electrode by running the camera supply wires with the rest of the connecting wires to the stimulation sites. The camera can also be embedded in the silicone body of the electrode. The size of the camera is not limited to the commercially available camera sizes, but can be any size suitable for being embedded in the electrode body. The characteristics of the camera optics can be provided by the vendor such that the camera has a large field of view (e.g., close to 180 degrees). In an alternative embodiment, the camera can utilize custom made lenses that are able to better adjust to cochlear imaging.

One method of custom-fabricated lenses for the visible and near infrared spectrums is through the use of droplets of OrmoComp®, a polymer, with a given curvature to achieve desired optical effects. Alternatively, a polymer other than OrmoComp® can be used. The formed droplets can be cured by ultraviolet (UV) light, resulting in miniature lenses.

Alternatively, the lenses can be printed using OrmoComp® as the ink in a 3D printer. Printing allows better control of the radius of curvature of the lenses. Once fabricated, the lenses can be mounted onto the chip of the camera. In alternative embodiments, the lenses can be formed by materials other than OrmoComp®).

In another illustrative embodiment, one or more polarizers can be placed between the lens and the camara chip. Using polarized illumination and a polarizer positioned in front of the camera provides the ability to cross-polarize the light and measure birefringent activity. Birefringent activity of the collagen, which is available in abundance in the cochlea, provides a direct measure of potential tissue damage.

Using a camera to acquire pictures and video in real-time provides a fast and efficient way to visualize cochlea structures, compared to other techniques that require 3D image reconstruction, such as CT or OCT. With a camera, real-time visualization feedback can be obtained quickly and easily, allowing for rapid assessment of the tissue or object features such as shape, size, texture, and color.

The video bridge chip of the proposed system is a specialized integrated circuit designed to enable the conversion of video signals between different formats and standards. Video bridge chips are commonly used in applications such as video game consoles, set-top boxes, and other multimedia devices that require the conversion of video signals. The video bridge chip plays a vital role in video signal processing by providing a seamless interface between different video standards and formats. It enables devices to support multiple video formats without the need for multiple connectors or adapters. The video bridge chip also supports advanced video features such as high dynamic range (HDR) and adaptive sync, which enhance the quality and performance of video output.

There are several video bridge chips available in the market. Among them, a suitable option for the microcamera OV6948 is the chip OV426. This chip provides an integrated analog-to-digital (A/D) data conversion using a built-in A/D converter (ADC), black level calibration (BLC), and a final digital video parallel output (DVP). The OV426 supports a standard interface to communicate with the system and manipulate the above-mentioned functions. During operation, the OV426 decodes the analog output from the OV6948 or a similar camera and digitizes it using the ADC. The generated digital signals can then be processed by a digital signal processor (DSP) and finally, standard DVP outputs are sent out to communicate with a DCMI block in the microcontroller. In an alternative embodiment, a different type of video bridge chip may be used and/or the video bridge chip can include different functionality.

The digital camera module interface, or DCMI, is a standard interface used in digital cameras and other imaging devices to transfer image data to a host system. The DCMI interface is critical to digital imaging because it allows for the efficient and reliable transfer of image data from the camera to the host system, such as a microcontroller or computer, for further processing or storage. DCMI is an important standard because it defines a uniform method for communication between cameras and host systems (microcontrollers), which promotes compatibility and ease of integration. The DCMI also supports various image data formats and resolutions, making it versatile and adaptable to a wide range of imaging applications.

There are several microcontrollers available in the market but not all of them have a DCMI module suitable for acquiring signals from cameras. In this regard, one option is the microcontroller STM32F429 from STMicroelectronics. This device allows users to develop rich applications easily with advanced graphic user interfaces. In alternative embodiments a different type of microcontroller can be used. Other alternatives including DCMI modules include the models STM32F407 and STM32746. In addition, the SPI/parallel ports are used so the microcontroller can communicate with a liquid crystal display (LCD) screen to visualize in real-time the pictures and videos captured by the microcamera. In alternative embodiments, a different type of microcontroller and/or DCMI can be used. Additionally, a different type of screen may be used such as light-emitting diode (LED), plasma, etc.

Signals acquired by the microcamera can be visualized on an LCD screen peripheral (or display). In an illustrative embodiment, the display device includes serial peripheral interface (SPI) ports and/or parallel ports to communicate with the microcontroller. The display can be selected to fulfil several requirements such as the pixels resolution to display the entire picture (e.g., at least 200×200 pixels). Furthermore, the sampling frequency of the display should be sufficient to capture and display the full frame rate of the camera sensor. For example, if the camera sensor captures images at 30 frames per second, then the display should have a sampling frequency of at least 30 Hz to display the full frame rate of the camera sensor.

An alternative to an LCD peripheral is to use a computer to display the pictures/videos. If this is the case, a UVC (USB Video Class) interface can be incorporated into the system. The UVC interface is a standard protocol for streaming video over a universal serial bus (USB) and is supported by most modern operating systems. By implementing the UVC interface in a computer, the camera sensor can be connected to the computer via a USB connection, allowing the video data to be transferred and displayed on the screen in real-time. The UVC interface also provides a standardized way of controlling the camera settings, such as resolution, frame rate, exposure, and white balance, which can be adjusted through the operating system's video capture application programming interface (API), which is a programming interface that allows software applications to capture and process video data from a camera sensor or other video source.

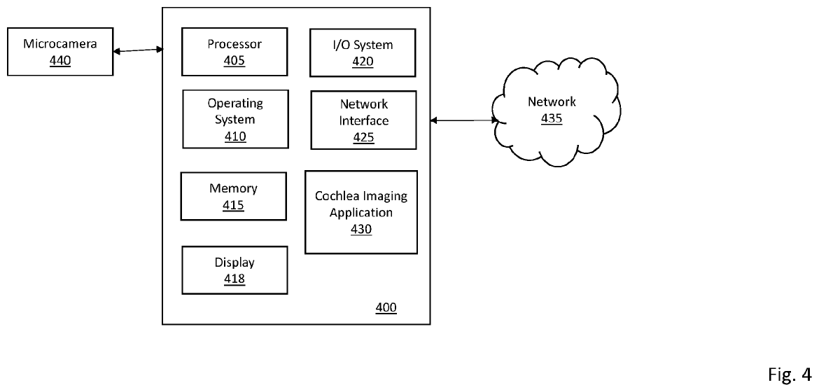

In one embodiment, the proposed system can also include additional computing components. The computing components can be incorporated into a dedicated computing system, a personal computing device (e.g., smartphone), a laptop computer, desktop computer, etc. FIG. 4 depicts a computing system 400 for monitoring a cochlea implant procedure in accordance with an illustrative embodiment. In one embodiment, at least a portion of the computing system 400 can be remote from the implanted microcamera, but in communication therewith through a network 435 or other form of wireless communication. The computing system 400 includes a processor 405, an operating system 410, a memory 415, a display 418, an input/output (I/O) system 420, a network interface 425, and a cochlea imaging application 430. In alternative embodiments, the computing system 400 may include fewer, additional, and/or different components. The components of the computing system 400 communicate with one another via one or more buses or any other interconnect system. As discussed, the computing system 400 can be any type of computing system (e.g., smartphone, tablet, laptop, desktop, etc.), including a dedicated standalone computing system that is designed to perform the cochlea imaging. In one embodiment, at least a portion of the computing system 400 may be incorporated into a microcamera 440 camera that is mounted to an electrode array being implanted into the cochlea.

The processor 405 can be in electrical communication with and used to control any of the system components described herein. For example, the processor 405 can be used to execute the cochlea imaging application 430, control the hardware (e.g., the microcamera 440), process image data, run algorithms, etc. The processor 405 can be any type of computer processor known in the art and can include a plurality of processors and/or a plurality of processing cores. The processor 405 can include a controller, a microcontroller, an audio processor, a graphics processing unit, a hardware accelerator, a digital signal processor, etc. Additionally, the processor 405 may be implemented as a complex instruction set computer processor, a reduced instruction set computer processor, an x86 instruction set computer processor, etc. The processor 405 is used to run the operating system 410, which can be any type of operating system.

The operating system 410 is stored in the memory 415, which is also used to store programs, received image/video data, network and communications data, peripheral component data, the cochlea imaging application 430, and other operating instructions. The memory 415 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 415 can be in the cloud to provide cloud storage for the system. Similarly, in one embodiment, any of the computing components described herein (e.g., the processor 405, etc.) can be implemented in the cloud such that the system can be run and controlled through cloud computing.

The I/O system 420 is the framework which enables users and peripheral devices to interact with the computing system 400. The display 418 can include a touch screen in some embodiments, and the touch screen can be part of the I/O system 420 that allows a user to make selections, control sub-systems, view results, etc. The display 418 can be any type of display, including a monitor, projector, etc., and can be used to present user interface screens, control screens, captured images, captured video, and other data to the user. The I/O system 420 can also include one or more speakers, one or more microphones, a keyboard, a mouse, one or more buttons or other controls, etc. that allow the user to interact with and control the computing system 400. The I/O system 420 also includes circuitry and a bus structure to interface with peripheral computing devices such as the microcamera, power sources, universal service bus (USB) devices, data acquisition cards, peripheral component interconnect express (PCIe) devices, serial advanced technology attachment (SATA) devices, high-definition multimedia interface (HDMI) devices, proprietary connection devices, etc.

The network interface 425 includes transceiver circuitry (e.g., a transmitter and a receiver) that allows the computing system 400 to transmit and receive data to/from other devices such as remote computing systems, servers, websites, cameras, etc. The network interface 425 enables communication through the network 435, which can be one or more communication networks. The network 435 can include a cable network, a fiber network, a cellular network, a wi-fi network, a landline telephone network, a microwave network, a satellite network, etc. The network interface 425 also includes circuitry to allow device-to-device communication such as Bluetooth® communication.

The cochlea imaging application 430 can include software and algorithms in the form of computer-readable instructions which, upon execution by the processor 405, performs any of the various operations described herein such as controlling the microcamera 440 to capture images, controlling the microcamera 440 to capture video, altering settings of the microcamera 440, analyzing the collected imagery, generating a warning if the captured imagery identifies a problem or potential problem, etc. The cochlea imaging application 430 can utilize the processor 405 and/or the memory 415 and/or the display 418 as discussed above. In an alternative implementation, the cochlea imaging application 430 can be remote or independent from the computing device 400, but in communication therewith.

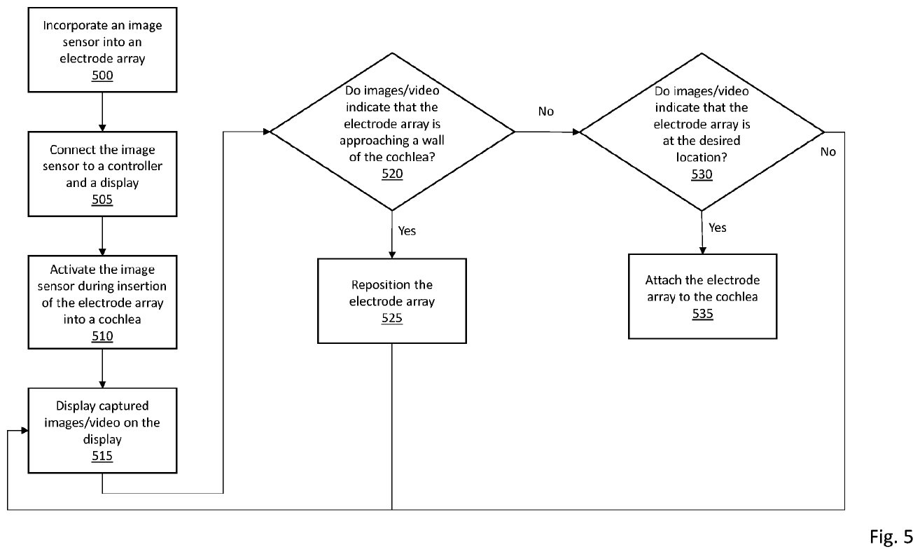

FIG. 5 is a flow diagram depicting operations performed to make and use a cochlear implant monitoring system in accordance with an illustrative embodiment. In alternative embodiments, fewer, additional, and/or different operations may be performed. Additionally, the use of a flow diagram is not meant to be limiting with respect to the order in which the operations are performed. In an operation 500, an image sensor is incorporated into an electrode array. The image sensor (or camera) can be incorporated into an electrode in one embodiment. Alternatively, the image sensor may be mounted to the electrode array independent of the electrodes that form the array. Any type of camera that is sized to fit within the cochlea conduit may be used. In another illustrative embodiment, the image sensor is mounted to the leading edge of the electrode array for optimal visibility during electrode array insertion.

In an operation 505, the image sensor is connected to a controller and a display. The controller can be any type of computer or other processing device that is able to control the image sensor. For example, the controller can be used to turn the image sensor on/off, to switch between image capture and video capture, to adjust the magnification, to control the frame rate, etc. The display can be any type of display or screen that is able to receive and present the captured images/videos from the image sensor. In an operation 510, the image sensor is activated during insertion of the electrode array into a cochlea of a patient. The image sensor can be activated via the controller, which can send wired and/or wireless control signals to the image sensor.

In an operation 515, the captured image/video is displayed on the display that is in communication with the image sensor. In an operation 520, based on the displayed image/video, a determination is made regarding whether the image/video indicates that the electrode array is approaching a wall of the cochlea. The determination can be made by a doctor or other individual performing the implant procedure in one embodiment. Alternatively, the determination can be made by the controller, which can use image processing to determine the position of the electrode array relative to the walls of the cochlea.

If it is determined in the operation 520 that the electrode array is approaching a wall of the cochlea, the electrode array is repositioned in an operation 525. The repositioning can be performed with the aid of the images/video from the image sensor. After repositioning the image sensor, the process returns to the operation 515 in which captured images/videos are displayed on the display. If it is determined in the operation 520 that the electrode array is not approaching a wall of the cochlea, a determination is made in an operation 530 regarding whether the electrode array is positioned at a desired location. If it is determined in the operation 530 that the electrode array is in the desired position, the electrode array is attached/mounted to the cochlea in an operation 535. Any techniques known in the art can be used to mount the electrode array to the cochlea of the patient. If it is determined in the operation 530 that the electrode array is not in the desired position, the process returns to the operation 515 in which additional captured images/video are displayed on the display.

Thus, described herein is a visualization system for real-time monitoring of electrode array insertion into the human cochlea as part of a cochlear implant procedure. The visualization system includes a microcamera located at a distal tip of the electrode array. The camera has a dimension suitable for the cochlea conduit (e.g., <1 mm). The color image sensor is capable of capturing video at up to 30 frames per second, providing smooth and seamless video playback. Despite its small size, the image sensor features advanced imaging technology, including a microlenses and back-illumination, which helps to improve image quality and sensitivity in low-light conditions. In addition, the camera chip is also designed to be power-efficient, with a low power consumption of just 2 milliwatts, making it suitable for use in battery-powered devices such as cochlear implants.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.” The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A cochlea implant monitoring system comprising:
a microcamera mounted to an electrode array of a cochlear implant;
a processor operatively coupled to the microcamera and configured to control the microcamera to capture images during insertion of the electrode array; and
a display operatively coupled to the processor and configured to display the captured images in real-time during insertion of the electrode array.

2. The system of claim 1, further comprising a video bridge chip coupled to the microcamera, wherein the video bridge chip comprises an integrated circuit that performs image processing.

3. The system of claim 2, wherein the video bridge chip includes an analog-to-digital converter (ADC) to convert captured analog data to digital data.

4. The system of claim 1, wherein the processor comprises a microcontroller that includes a digital camera module interface (DCMI).

5. The system of claim 1, wherein the processor comprises a microcontroller that includes one or more of a parallel interface and a serial peripheral interface.

6. The system of claim 1, wherein the microcamera is mounted to a distal end of the electrode array.

7. The system of claim 1, wherein a sampling frequency of the display is greater than or equal to a frame capture rate of the microcamera.

8. The system of claim 1, wherein the display comprises a liquid crystal display (LCD).

9. The system of claim 1, further comprising one or more microlenses mounted to the microcamera, wherein the one or more microlenses are produced by curing one or more droplets of a polymer with ultraviolet (UV) light.

10. The system of claim 1, further comprising one or more microlenses mounted to the microcamera, wherein the one or more microlenses are in the form of a printed transparent polymer.

11. The system of claim 1, further comprising:
a polarizer positioned in front of a camera chip of the microcamera; and
a light source that emits polarized light toward the camera chip such that birefringent activity can be monitored by the processor.

12. The system of claim 1, further comprising:
a polarizer positioned in front of a camera chip of the microcamera; and
a waveguide in the form of an optical fiber or a polymer, wherein the waveguide directs polarized light toward the camera chip such that birefringent activity can be monitored by the processor.

13. A method of monitoring a cochlear implant during insertion, the method comprising:
controlling, by a processor, a microcamera mounted to an electrode array of the cochlear implant;
capturing, by the microcamera, images during insertion of the electrode array into a cochlea; and
displaying, by a display operatively coupled to the processor, the captured images in real-time during insertion of the electrode array.

14. The method of claim 13, further comprising controlling a frame capture rate of the microcamera to be less than or equal to a sampling frequency of the display.

15. The method of claim 13, further comprising emitting polarized light toward the microcamera.

16. The method of claim 15, wherein the processor monitors birefringent activity that results from the polarized light.

17. The method of claim 15, wherein the polarized light is emitted from a light source proximate to the microcamera.

18. The method of claim 15, wherein the polarized light is emitted from a waveguide in the form of an optical fiber or a polymer.

19. The method of claim 13, further comprising mounting one or more microlenses to the microcamera, wherein the one or more microlenses are produced by curing one or more droplets of a polymer with ultraviolet (UV) light.

20. The method of claim 13, wherein the microcamera is incorporated into an electrode of the electrode array.
